(19) United States
(12) Patent Application Publication
(10) Pub. No.: US 2009/0226517 A1
(43) Pub. Date: Sep. 10, 2009

(54) PHARMACEUTICAL FORMULATIONS COMPRISING DULOXETINE

(76) Inventors: Vinita Umashankar Vyas, Bhopal (IN); Nagaraju Nagesh, Hyderabad (IN); Pavan Kumar Mittapalli, Guntur (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/366,246

(22) Filed: Feb. 5, 2009

Related U.S. Application Data
(60) Provisional application No. 61/047,116, filed on Apr. 23, 2008.

(30) Foreign Application Priority Data
Feb. 6, 2008 (IN) 316/CHE/2008

(51) Int. Cl. A61K 9/24; B05D 3/00; A61P 25/24

(52) U.S. Cl. 424/472; 427/2.14

(57) ABSTRACT

A pharmaceutical formulation comprising: a core particle comprising duloxetine or its salts; a separating layer; and an enteric layer disposed over the separating layer; wherein the formulation comprises at least one amino acid, a plasticizer, or both, in at least one of the core, the separating layer, and the enteric layer.

### INTRODUCTION

The present invention relates to pharmaceutical formulations comprising duloxetine, its pharmaceutically acceptable derivatives, and mixtures thereof. The invention further relates to processes for preparing the formulations.

Duloxetine hydrochloride has a chemical name (+)-(S)—N-methyl-γ-(1-naphthyloxy)-2-thiophenepropylamine hydrochloride and is represented by structural Formula I.

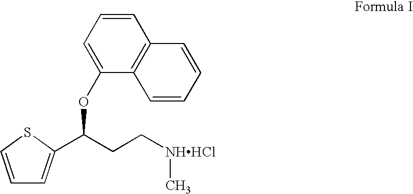

Duloxetine is selective serotonin and norepinephrine reuptake inhibitor (SSNRI) used in major depressive disorders, diabetic peripheral neuropathic pain and generalized anxiety disorders. In Europe, it is also approved for stress urinary incontinence. Duloxetine-containing products have been marketed as CYMBALTA®, YENTREVE®, XERISTER™ and ARICLAIM™ in the dose strengths of 20, 30 and 60 mg of duloxetine base (22.4, 33.7 and 67.3 mg of duloxetine hydrochloride) and the dosage varies from 20-60 mg once- or twice-daily, depending upon the particular disorder to be treated.

Duloxetine hydrochloride is a white to slightly brownish white solid, slightly soluble in water. Duloxetine is an acid-sensitive drug that readily reacts in the gastric environment. As the site of absorption for duloxetine is the intestine, it is important to protect the drug before it reaches the intestine. An enteric coating is the most commonly used technology for formulation of such acid-sensitive drugs.

Duloxetine is disclosed in U.S. Pat. No. 5,023,269. U.S. Pat. Nos. 5,910,319 and 5,508,276, and U.S. Patent Application Publication Nos. 2006/0165776 and 2007/0167513, disclose pharmaceutical formulations of duloxetine.

International Application Publication No. WO 2005/065726 discloses pharmaceutical dosage forms comprising components that have a tendency to chemically interact with coatings having acidic functional groups, and more particularly to dosage forms containing components that interact with enteric coating materials.

### SUMMARY

The present invention relates to pharmaceutical formulations comprising an acid-sensitive drug or its pharmaceutically acceptable salts.

The present invention relates to pharmaceutical formulations comprising duloxetine or its pharmaceutically acceptable salts.

In an embodiment, the present invention provides solid dosage forms comprising:

(1) An inert core.

(2) Optionally, a seal coating upon the inert core.

(3) Drug layering over the core comprising at least one acid-sensitive active substance.

(4) A subcoating thereupon, optionally comprising a substance that reacts with acidic functional groups.

(5) An acidic functional group-containing enteric coating over the subcoating, optionally having a plasticizer.

(6) An optional outer finishing layer.

In another embodiment, the invention provides solid dosage forms comprising:

(1) An inert core.

(2) Optionally, a seal coating upon the inert core.

(3) A drug layer over the core comprising at least one acid-sensitive active substance.

(4) A subcoating that optionally comprises at least one reactive substance such as an α-amino acid.

(5) An acidic functional group-containing enteric coating over the subcoating, optionally having a plasticizer.

(6) An optional outer finishing layer.

In yet another embodiment, the invention provides solid dosage forms comprising:

(1) An acid-sensitive drug substance in a core.

(2) A seal coating upon the core comprising at least one chemically reactive substance.

(3) An acidic functional group-containing enteric coating over the subcoating, containing a water-insoluble plasticizer.

(4) An optional outer finishing layer.

In an embodiment, formulations of the present invention comprise multi-particulate systems comprising a plurality of beads, pellets, particles, granules, or minitablets that can be compressed to form tablets, or can be filled into capsules.

In another embodiment, the formulations of the present invention are monolithic, comprising a single unit dosage form.

In an embodiment, the invention includes duloxetine or a salt thereof as the acid-sensitive active substance.

In another embodiment the invention includes glycine as a chemically reactive substance.

In one aspect, the present invention provides enteric coated pharmaceutical formulations comprising duloxetine or any of its salts, optionally an excipient having a tendency to chemically interact with enteric coating material, and/or optionally a water insoluble plasticizer.

In another embodiment, the composition contains a water-insoluble plasticizer, which can be accommodated within any of the coating layers, viz. subcoating, enteric coating and/or seal coating.

In one of the embodiments, the formulations contain a combination of water-soluble and water-insoluble plasticizers.

In one aspect of the present invention, pharmaceutical formulations include a plasticizer in the range from about 0.1 to about 20%, or from about 0.5 to about 10%, by weight of the coating layer.

In an embodiment, the present invention relates to pharmaceutical formulations of duloxetine or its salts wherein dissolution testing shows less than about 10% of contained duloxetine being released within the first 120 minutes after immersion into 0.1 N HCl. The release of the drug occurs thereafter, in the range of about 50% to about 90%, or about 55% to about 75%, of drug dissolving within about 15 minutes, and about 90% to about 100% of the drug dissolving within about 90 minutes, during immersion in higher-pH buffer.

In one of the embodiments, the invention relates to processes for preparing pharmaceutical formulations of duloxetine.

In one of the embodiments, the invention relates to methods of using pharmaceutical formulations of the present invention in the treatment of major depressive disorders.

### DETAILED DESCRIPTION

The present invention relates to pharmaceutical formulations comprising an acid-sensitive drug or its pharmaceutically acceptable derivatives.

The present invention relates to pharmaceutical formulations comprising duloxetine or its pharmaceutically acceptable derivatives.

The invention also relates to processes for preparing the formulations and their methods of use.

Pharmaceutically acceptable derivatives of duloxetine include but are not limited to salts, polymorphs, solvates, esters, hydrates, enantiomers, racemic mixtures, and combinations thereof.

In the present invention, coatings that contain high amounts of acidic groups can be used. Acidic polymers, which have free carboxylic groups, are widely used in pharmaceutical formulations, especially for enteric coating. An enteric coating is an element of the pharmaceutical dosage form in embodiments of the present invention. One advantage of the present invention is the ability to use virtually any polymer for this coating, as compared with the teaching of U.S. Pat. No. 5,910,319 that the concentration of acidic groups in the coating must be limited.

Duloxetine belongs to BCS class II and thus has limited aqueous solubility. There are instances where the rate of dissolution of a poorly soluble drug is a rate-limiting factor in its absorption by the body. It is recognized that such drugs may be more readily bioavailable if administered in a finely divided state. The rate of dissolution depends on factors including particle sizes (or particle surface areas, which can be related to particle sizes). Particle sizes also can affect how freely crystals or other powdered forms of a drug will flow, which has consequences in the production processing of pharmaceutical products containing the drug. Solubility will increase with decreasing sizes of solute particles because of the additional surface energy. The effect of the particle size on solubility constant can be quantified as follows:

Log*K_{A}=Log*K_{A→0}+(2γAₘ÷3 ln(10)RT)

where *K_{A} is the solubility constant for the solute particles with the molar surface area A, K_{A→0} is the solubility constant for substance with molar surface area tending to zero (i.e., when the particles are large), γ is the surface tension of the solute particle in the solvent, Aₘ is the molar surface area of the solute (in m²/mol), R is the universal gas constant, and T is the absolute temperature.

The fractions of particles with different dimensions that exist in a powder is called the particle size distribution. It is represented in certain ways. Particle size is the maximum dimension of a particle, normally expressed in units of μm. The D₁₀, D₅₀ and D₉₀ values are useful ways for indicating a particle size distribution. D₉₀ refers to at least 90 volume percent of the particles having a size smaller than the specified value. Likewise D₁₀ refers to 10 volume percent of the particles having a size smaller than the specified value. D₅₀ refers to 50 volume percent of the particles having a size smaller than the specified value.

In an embodiment the invention includes pharmaceutical formulations comprising duloxetine or its salts with a specific particle size distribution, wherein: D₁₀ is less than about 50 μm, or less than about 20 μm; D₅₀ is less than about 100 μm, or less than about 50 μm; and D₉₀ is less than about 300 μm, or less than about 200 μm.

For purposes of the present invention, an “enteric coating” is a hydrophobic layer that surrounds inner components of a pharmaceutical dosage, which inner components include at least a portion of the total active drug. The enteric coating resists decomposition by gastric juices and thereby protects the inner components from interaction with acidic substances that are present in the stomach. Frequently the enteric coating itself is an acidic substance and will be decomposed or removed upon exposure to a less acidic environment. Various commercially available enteric materials are designated to undergo decomposition or removal when exposed to certain pH conditions that are associated with specific regions of the digestive tract of a human: pH between about 3.5 and 6 in the duodenum; pH between about 6 and 7 in the jejunum; pH between about 7 and 7.5 in the ileum; and pH between about 7 and 8 in the colon. In most cases, complete removal of enteric coating is not necessary for obtaining a desired drug release, as any substantial discontinuity in the coating will permit fluid ingress for interaction with the inner components. The enteric coat can optionally include a water-insoluble material that prevents penetration of water into the core or the drug layer and solubilize the drug before the dosage form reaches the lower gastrointestinal tract.

Selection of the enteric material becomes more critical when the active has a tendency to chemically interact with the enteric coating materials. This may result in slowly soluble or even insoluble coatings that impair the complete release of active ingredient from the dosage form. Some widely used antidepressants, including duloxetine, that are acid-sensitive have been reported to interact with enteric coating materials. The formulations involving enteric coatings essentially include a coating polymer and a plasticizer. Many plasticizers are high boiling point organic solvents and are included to impart plasticity or fluidity and to increase flexibility of the enteric layer.

The water insoluble material would prevent penetration of water into the pharmaceutical composition and thereby prevent dissolution of the drug before the enteric coating is dissolved at least partially. This would prevent interactions of the drug with the enteric polymer, as presence of moisture is responsible for catalyzing the reaction.

As described above, the commonly used enteric coating materials are polymers having acidic functional groups. For the purpose of this invention, the terms “chemically reactive substance”, “reactive substance” and “chemically reactive component” represent a component of a pharmaceutical dosage form that undergoes a competitive chemical reaction with carboxylic groups in the enteric coating to prevent the interaction between inner components of the dosage form and the enteric coating polymer. Chemically reactive substances are present in a subcoating upon the core; or in core; or in both the core and the subcoating, the subcoating being in contact with an enteric coating.

The enteric coating includes an enteric polymer and a plasticizer. Plasticizers will improve the physical/mechanical properties of films, including the flexibility, tensile strength and Young's modulus. The most commonly used plasticizers in pharmaceutical practice include, but are not limited to, glycerin, acetylated monoglyceride, propylene glycols, polyethylene glycols (e.g., PEG 400 and PEG 8000), triacetin, triethyl citrate (TEC), acetyl tributyl citrate (ATBC), acetyl triethyl citrate (ATEC), dibutyl phthalate, and dibutyl subacate. Citrate esters have been used extensively in the preparation of pharmaceutical dosage forms and display good efficiency, stability, permanence and polymer/plasticizer compatibility.

Duloxetine undergoes many degradation reactions and the most common degradation is by hydrolysis. α-Naphthol and 4-naphthol duloxetine impurities are the major degradants formed by hydrolysis. Some degradation products of duloxetine include:

1) “α-Naphthol” having a chemical name 1-hydroxynapthalene and represented by Formula II.

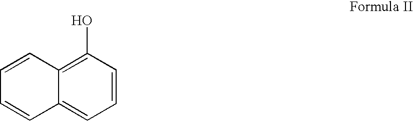

2) “4-naphthol duloxetine” having a chemical name 4-(3-methylamino-1-thiophen-2-yl-propyl)-naphthalene-1-ol hydrochloride and represented by Formula III.

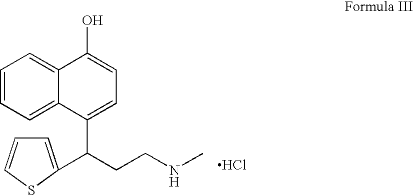

3) “3-Acetyl duloxetine” having a chemical name (+)-N-methyl-3-(1-naphthalenyloxy)-3-mine hydrochloride and represented by Formula IV.

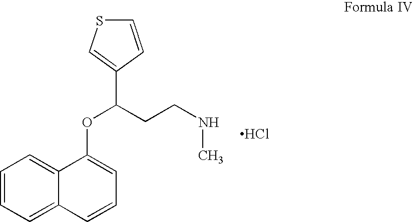

In an embodiment the invention relates to analytical methods for analysis of duloxetine-related impurities using high performance liquid chromatography (HPLC), wherein a method comprises:

Buffer solution: 1.36 g of potassium dihydrogen phosphate is dissolved in 1000 mL of water, add 10 mL of triethylamine and mix well. Adjust the pH to 4.0 with diluted ortho phosphoric acid and filter through 0.45 μm nylon 66 membrane filter.

Mobile phase A: Mix buffer (pH 4.0), tetrahydrofuran and methanol in the volume ratio of 73:20:7, respectively. Degas in a sonicator for about 10 minutes.

Diluent: Mix Milli-Q water and methanol in the volume ratio of 10:90, respectively.

Chromatographic System:
(1) The liquid chromatograph is equipped with a 215 nm UV detector.
(2) Column: 150 mm×4.6 mm, 5 μm, Symmetry C-8 or equivalent.
(3) Column temperature: 40° C.
(4) Flow rate: 1.0 mL per minute.
(5) Injection volume: 10 μL.
(6) Run time: For blank, system suitability and test preparation about 70 minutes. For standard preparation about 20 minutes.

Preparation of Test Sample:

10 capsules are emptied and powder equivalent to 56 mg of duloxetine hydrochloride is placed into a 100 mL volumetric flask. Then 70 mL of diluent is added and the mixture subjected to sonication for 30 minutes with intermittent shaking and the volumetric flask is filled to volume with diluent. Finally a portion of the solution is subjected to centrifugation at 3000 RPM for about 10 minutes before injection.

Representative relative retention times (RRT, where duloxetine=1), limits of detection (LOD), and limits of quantification (LOQ) of various impurities are tabulated below, where LOD and LOQ values are percent by weight of the label duloxetine content.

| Impurity | RRT | LOD | LOQ |
|---|---|---|---|
| 4-Naphthol duloxetine | 0.62 | 0.003 | 0.014 |
| 3-Acetyl duloxetine | 1.21 | 0.005 | 0.023 |
| α-Naphthol | 3.11 | 0.006 | 0.026 |

In embodiments the present invention includes pharmaceutical formulations comprising duloxetine or its salts, wherein the formulations comprise less than about 2%, or less than about 1%, or less than about 0.5%, by weight of 4-naphthol duloxetine. In an embodiment the present invention includes pharmaceutical formulations comprising duloxetine or its salts, wherein the formulations comprise less than about 2%, or less than about 1%, or less than about 0.5%, by weight of 3-acetyl duloxetine.

In embodiments the present invention includes pharmaceutical formulations comprising duloxetine or its salts, wherein the formulations comprise less than about 2%, or less than about 1%, or less than about 0.5%, by weight of α-naphthol. Further embodiments include pharmaceutical formulations comprising duloxetine or its salt, wherein formulation comprises less than 2%, or less than 1%, by weight of total degradation products. All of the foregoing impurity contents are expressed as percentages of the label drug content in the pharmaceutical formulations.

In an embodiment the invention includes stable pharmaceutical formulations comprising duloxetine or its salts.

An embodiment of a pharmaceutical dosage form of this invention comprises at least: (1) a core containing one or more drug substances and/or one or more excipients; (2) alternatively an inert core coated with a drug layer containing acid-sensitive drug or its salt and one or more excipients; (3) optionally a chemical reactive substance-containing subcoating over the core or over the drug layer; (4) an enteric coating layered over the subcoating; and (5) optionally a film or other coating over the enteric coating. Optionally, the composition contains a water-insoluble excipient that can be accommodated within any or all of the three coatings over the drug layer. Components of the core can sometimes be referred to herein as “inner components.”

In an embodiment, the invention includes pharmaceutical formulations comprising duloxetine or a salt thereof with defined particle size distribution and at least one pharmaceutically acceptable excipient.

The pharmaceutical formulations of the present invention include solid dosage forms such as tablets, capsules, granules, pellets, pills, etc.

In one embodiment, the formulations of the present invention include higher amounts of duloxetine hydrochloride, such as in concentrations more than about 4% by weight, or more than about 10% by weight.

The core may be in any of a number of physical forms, such as pellets, granules, beads, minitablets and tablets, such as are conventionally used for the oral administration of pharmaceutical substances. Pellets can be prepared using techniques such as extrusion and spheronization, by coating non-pareil seeds, by melt pelletization, or by other pelletization processes. Useful non-pareil seeds can be prepared from starch and sucrose, using techniques that are well known in the art. Tablets and minitablets can be prepared by customary compression techniques with or without involving a prior granulation step, such as wet granulation, dry granulation, or melt granulation. If desired, minitablets, granules, pellets, beads, and the like that are prepared according to this invention can be filled into capsules to produce a final dosage form, for ease of administration.

The core of a formulation is normally prepared by mixing an active ingredient or active ingredients with a desired combination of excipient ingredients such as diluents or fillers, surfactants, disintegrants, binders, lubricants, plasticizers and optionally a chemically reactive substance. When a chemically reactive substance is present in the core, its concentration typically will be about 0.5 to about 20 percent, or about 1 to about 15 percent, or about 2 to about 8 percent, of the total weight of the core. In general, the core comprises about 10 to about 80 percent, or about 30 to about 70 percent, or about 45 to about 65 percent, of the final formulation.

The core can be coated with a seal coating. Excipients used for seal coating include, but are not limited to, cellulose derivatives such as hydroxymethyl cellulose, hydroxypropyl methylcellulose, and hydroxypropyl cellulose.

The subcoating layer may comprise one or more excipients. Suitable inert excipients for use as a subcoating material include, without limitation thereto, carageenan, ethylcelluloses, hydroxypropyl methylcelluloses, hydroxypropyl celluloses, methylcelluloses, carboxymethyl celluloses, hydroxymethyl celluloses, hydroxyethyl celluloses, polyethylene glycols, polyvinyl alcohols, and xanthan gum.

Suitable chemically reactive components include amino acids, particularly but not limited to naturally occurring α-amino acids that are recognized as being safe for ingestion. Examples of those amino acids are glycine, alanine, valine, leucine, isolucine, serine, threonine, methionine, cysteine, aspartic acid, asparagine, glutamic acid, glutamine, arginine, lysine, histidine, phenylalanine, tyrosine, tryptophan and proline. In general, amino acids having lower molecular weights are preferred. Glycine, for example, has been found to be useful in the invention.

The chemically reactive component can conveniently be included in an aqueous-based subcoating comprising a cellulosic polymer or derivative thereof, which is conventionally used in the pharmaceutical industry for forming water-soluble or water-dispersible films. Some of the polymers include, without limitation thereto, cellulose derivatives such as methylcelluloses, ethylcelluloses, hydroxypropyl methylcelluloses, hydroxypropyl celluloses, and combinations thereof. Acrylics, such as methacrylate and methyl methacrylate copolymers, and vinyls, such as polyvinyl alcohols, can be used for subcoating. Other polymers, such as a poly(N-vinyl-2-pyrrolidone), (“PVP” or “povidone”) and derivatives such as copolymers of N-vinyl-2-pyrrolidone and vinyl acetate (“copovidone”) are also useful.

Typically, a subcoating will comprise about 1 to about 60 percent, or about 5 to about 45 percent, or about 7 to about 30 percent, of the total weight of the final dosage form.

The amount of chemically reactive component incorporated in the subcoating is typically about 10 to about 60 percent, or about 15 to about 30 percent, by weight of the total subcoating. Expressed as a percentage of the final dosage form, the chemically reactive component therefore typically comprises about 0.1 to about 20 percent, or about 0.5 to about 15 percent, or about 0.7 to about 10 percent, by weight of the dosage form.

The amount of subcoating will depend somewhat on the nature of the core particles, as smaller cores such as pellets and granules usually will have a large total surface area and require a larger quantity of coating material to obtain coverage, than will tablets. For this reason, the percentages above are only general guidelines.

The subcoating may further include one or a combination of other commonly used functional ingredients such as diluents, binders, glidants or anti-adherents.

Various useful diluents include but are not limited to starches, lactose, sucrose, mannitol, Pearlitol™ SD 200, cellulose derivatives, confectioners sugar and the like. Different grades of lactose include but are not limited to lactose monohydrate, lactose DT (direct tableting), lactose anhydrous, Flowlac™ (available from Meggle Products), Pharmatose™ (available from DMV) and others. Different grades of starches include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline cellulose and powdered cellulose. Examples of crystalline cellulose products include but are not limited to CEOLUS™ KG801, Avicel™ PH101, PH102, PH301, PH302 and PH-F20, microcrystalline cellulose 114, and microcrystalline cellulose 112. Other useful diluents include but are not limited to carmellose, sugar alcohols such as mannitol, sorbitol, and xylitol, calcium carbonate, magnesium carbonate, dibasic calcium phosphate, and tribasic calcium phosphate.

Various useful binders include but are not limited to hydroxypropylcelluloses (Klucel™-LF), hydroxypropyl methylcelluloses or hypromelloses (Methocel™), polyvinylpyrrolidones or povidones (PVP-K25, PVP-K29, PVP-K30, PVP-K90), Plasdone™ S 630 (copovidone), powdered acacia, gelatin, guar gum, carbomers (e.g. Carbopol™), methylcelluloses, polymethacrylates, and starches.

Various useful glidants or anti-adherents include but are not limited to talc, silica derivatives, colloidal silicon dioxide and the like, including mixtures thereof.

Application of subcoatings to the particles generally proceeds according to accepted practice, such as by spraying onto particles present in a rotating pan, using a fluidized bed coating apparatus, and the like. The subcoating should be at least substantially continuous over the core, having no discontinuities that could permit direct physical contact between the core and the enteric coating.

Another useful subcoating material is zein, a prolamine that is insoluble in both water and alcohols, but soluble in mixtures thereof. A zein coating is a moisture-resistant coating and shows excellent storage stability. The chemically reactive component can be dissolved in a zein solution, and applied to core particles in the usual manner. Zein, derived from corn and in grades acceptable for pharmaceutical uses, is commercially available from Freeman Industries LLC of Tuckahoe, N.Y., U.S.A. as Zein F6000. The zein can be used alone, or in combination with a polymer or mixture of polymers.

Examples of useful enteric coating materials include the polymethacrylates sold by Evonik Industries AG, Darmstadt, Germany using the EUDRAGIT trademark. The EUDRAGIT L 100-55 and L 30 D-55 products are 1:1 copolymers of methacrylic acid and ethyl acrylate that dissolve at pH values above about 5.5. EUDRAGIT S 100 is a 1:1 copolymer of methacrylic acid and methyl methacrylate that dissolves at pH above about 6. Mixtures of EUDRAGIT L 100 and EUDRAGIT S 100, a 1:2 copolymer of methacrylic acid and methyl methacrylate, dissolved at pH above about 6 to 6.5. EUDRAGIT S 100 alone dissolves at pH above about 6.5-7.5. Chemically related enteric coating polymers are sold by Eastman Chemical Company of Kingsport, Tenn. USA under the trademark EASTCRYL, and by BASF of Ludwigshafen, Germany under the trademark KOLLICOAT.

“HPMCP” (hydroxypropyl methylcellulose phthalate, or hypromellose phthalate) sold as HP-55 by Shin-Etsu Chemical Co. Ltd., of Tokyo, Japan, is another useful enteric coating polymer. This material is almost universally accepted by regulatory authorities as an enteric coating substance. By varying the phthalate content, the pH for dissolution of the coating can be controlled, generally to values between about 5 and about 5.5. This product has about 31 percent phthalyl content and is soluble in McIlvaine's buffer solution of pH 5.5 or greater. Also available from this source is the HP-50 product that has a phthalyl content about 24 percent, and solubility at pH 5 and above.

Other types of enteric coating polymers are known, and can be used for purposes of this invention. These include polyvinylacetate phthalates, cellulose acetate trimelliates, carboxymethylethyl celluloses, hydroxymethyl cellulose acetate succinates and cellulose acetate phthalates. The polymers are commercially available, from several sources.

Many commercial products are sold as ready-to-use mixtures of the polymer, a plasticizer, and other desired functional components such as glidants and/or pigments. These products can be simply dissolved or dispersed in a suitable solvent and applied to particles containing the drug substance.

The enteric coatings may be applied in a powder form or from an aqueous or organic solution or dispersion. Typically subcoated core pellets, granules, tablets etc. are coated with a fluid dispersion or solution that contains the enteric polymer, since this facilitates a more uniform coating. The enteric coating layer is applied to the subcoated cores using conventional coating techniques such as pan coating or fluidized bed coating using any of the previously mentioned polymers, and others. In the finished dosage form, the quantity of enteric coating is typically about 5 to about 30 percent, or about 10 to about 25 percent, by weight.

Solid property-modifying components such as talc can also be incorporated into an enteric coating solution or dispersion, as is well-known in the art.

The enteric coating is frequently applied as a solution of enteric polymer in organic solvents like acetone, dichloromethane and isopropyl alcohol, and combinations thereof. In another embodiment of the invention, a suspension of enteric coated polymer can be applied over the subcoating, provided the suspension remains homogenous. Application of the enteric layer to the subcoated product can be conducted using fluid bed type equipment or in a conventional rotating coating pan with simultaneous spraying of enteric polymer solution or suspension and warm air drying. Temperature of the drying air and the temperature of the circulating mass of pellets should be kept in the ranges advised by the manufacturer of the particular enteric polymer being used.

A finishing layer over the enteric layer is not necessary in every case, but generally improves the elegance of the product, as well as its handling, storage and flow characteristics and may provide further benefits. The most simple finishing layer is simply a small amount, such as about 1% by weight, of an anti-static ingredient such as talc or silicon dioxide, simply dusted on the surface of the particles.

As is known in the art, a finishing coating of the dosage forms such as tablets can be a film coating that improves the surface properties and facilitates imprinting of identifying information. Although less preferable from the manufacturing convenience perspective, a sugar coating that is applied in the usual manner can be used for the same purposes.

For purposes of present invention, the fillers may include but are not restricted to sucrose, dextrose, lactose, fructose, microcrystalline cellulose, calcium carbonate, sorbitol, xylitol, isomalt, gelatin and starches.

An opacifier like titanium dioxide may also be present in an amount ranging from about 10% (w/w) to about 20% (w/w), based on the total weight of the coating.

Anti-adhesives are frequently used in film coating processes to avoid sticking effects during film formation and drying. An example of an anti-adhesive for this purpose is talc.

Suitable polishing agents include polyethylene glycols of various molecular weights or mixtures thereof, talc, surfactants (e.g. glycerol monostearate and poloxamers), fatty alcohols (e.g., stearyl alcohol, cetyl alcohol, lauryl alcohol and myristyl alcohol) and waxes (e.g., carnauba wax, candelilla wax and white wax).

In embodiments, the pharmaceutical formulations of the present invention exhibit improved storage stability over a commercial reference product during storage under accelerated conditions.

Pharmaceutical products can be tested for their drug release characteristics, such as using the procedure described in Test 711 “Dissolution,” United States Pharmacopeia 29, United States Pharmacopeial Convention, Inc., Rockville, Md., pages 2673-2682, 2005 (“USP”).

Following are examples of pharmaceutical formulations incorporating active ingredients that are sensitive to enteric coating polymers. The examples are intended only to illustrate certain specific aspects and embodiments of the invention, and are not to be construed as limiting the scope of the invention in any manner. In the examples, solvents that are used in processing but are not present in the final dosage form are included in ingredient listings.

### Example 1

### Duloxetine Hydrochloride-Containing Enteric-Coated Pellets

| | |
|---|---|
| Ingredient | Weight Percent |

DRUG LAYERING

| | |
|---|---|
| Sugar spheres (#30-#35 mesh) | 26.71 |
| Duloxetine hydrochloride | 27.36 |
| Hydroxypropyl methylcellulose 5 cps | 4.06 |
| Glycine | 10.16 |
| Water* | q.s. |

SUBCOATING

| | |
|---|---|
| Hydroxypropyl methylcellulose 5 cps | 3.25 |
| Sucrose | 12.15 |
| Talc | 3.29 |
| Water* | q.s. |

ENTERIC COATING

| | |
|---|---|
| Hypromellose phthalate HP 55 | 10.41 |
| Talc | 1.58 |
| Triethyl citrate | 1.02 |
| Isopropyl alcohol* | q.s. |
| Methylene chloride* | q.s. |

LUBRICATION

| | |
|---|---|
| Duloxetine hydrochloride-containing | 99.72 |
| enteric coated pellets | |
| Talc | 0.28 |

*Evaporates during processing.

Manufacturing Process:

Drug Coating:

1) HPMC 5 cps was dissolved in hot water maintained at about 60-70° C. The solution was stirred and allowed to cool to room temperature.

2) Glycine and duloxetine hydrochloride were added to the polymer solution under continuous stirring.

3) Drug-loading dispersion was coated onto sugar spheres using the Wurster technique in a fluid-bed processor.

4) Drug-loaded pellets were dried and sifted. The #18-#24 mesh fraction of drug-coated pellets was collected.

Subcoating:

5) HPMC 5 cps, sucrose and talc were dispersed in water and a uniform and smooth sub-coating dispersion was formed.

6) Sub-coating dispersion was sprayed on drug-loaded pellets of step 4) using the Wurster technique in a fluid-bed processor.

7) Sub-coated pellets were dried and sifted. The #18-#24 mesh fraction of sub-coated pellets was collected.

Enteric Coating:

8) Hypromellose phthalate (HP 55) was dispersed in isopropyl alcohol.

9) To the polymer dispersion, methylene chloride and triethyl citrate were added under continuous stirring.

10) Talc was milled with isopropyl alcohol in a colloid mill and the dispersion was added to the polymer solution under stirring.

11) Enteric-coated dispersion was sprayed onto sub-coated pellets using the Wurster technique in a fluid-bed processor.

12) Enteric-coated pellets were dried and sifted. The #16-#24 mesh fraction of enteric-coated pellets was collected.

Lubrication

13) Enteric-coated pellets from xii) and talc were transferred into a bottom spray bowl and fluidized at low fluidization.

Capsule Filling:

14) 82.14, 123.4 and 246.7 mg of lubricated enteric-coated pellets were filled into size “4,” size “3,” and size “1” capsules, respectively, to achieve duloxetine hydrochloride 20, 30, and 60 mg capsules.

Capsules of Example 1 (“T”) and Cymbalta® capsules (“R”) were tested for drug release characteristics using the USP procedure and 1 L of 0.1 N HCl for the first 2 hours, followed by 1 L of pH 6.8 phosphate buffer, with USP Type I apparatus at 100 RPM stirring. The data are given below:

| Cumulative % of Drug Released | | |
|---|---|---|
| Capsule | | |
| 60 mg | 30 mg | 20 mg |

| | | | | | |
|---|---|---|---|---|---|
| T | R | T | R | T | R |

0.1 N HCl

| | | | | | | |
|---|---|---|---|---|---|---|
| 2 hours | 5 | 2 | 4 | 2 | 3 | 2 |

pH 6.8 Buffer

| | | | | | | |
|---|---|---|---|---|---|---|
| 15 minutes | 83 | 48 | 82 | 43 | 77 | 38 |
| 30 minutes | 88 | 78 | 90 | 83 | 88 | 78 |
| 45 minutes | 90 | 90 | 93 | 94 | 90 | 90 |
| 60 minutes | 92 | 96 | 95 | 97 | 92 | 93 |
| 90 minutes | 94 | 100 | 96 | 98 | 94 | 94 |

Example 1 capsules and CYMBALTA capsules, both containing 60 mg of duloxetine, were packaged in closed high density polyethylene containers and exposed to accelerated stability testing conditions (40° C. and 75% relative humidity) for 3 months. Samples were tested for degradation impurities (% by weight of label drug content), and water (% w/w by Karl Fischer method), and the data are given below.

| | CYMBALTA ® | | Example 1 | | | |
|---|---|---|---|---|---|---|
| Impurity | Initial | 3 Months | Initial | 1 Month | 2 Months | 3 Months |
| α-Napthol | 0.008 | ND** | BLD* | BLD | BLD | BLD |
| 4-Napthol duloxetine | ND | 0.01 | 0.023 | 0.027 | 0.025 | 0.025 |
| 3-Acetyl duloxetine | 0.126 | 0.115 | 0.041 | 0.047 | 0.038 | 0.022 |
| Highest unidentified impurity | 0.098 | 0.096 | 0.064 | 0.063 | 0.063 | 0.073 |
| Total impurities | 0.25 | 0.221 | 0.174 | 0.146 | 0.137 | 0.165 |
| Water | — | 1.29 | 1.2 | 1.5 | 1.7 | 1.4 |

*BLD: Below limit of detection.
**ND: Not detected.

Capsules were further evaluated in a two-way crossover pharmacokinetic study, involving administration of the 60 mg duloxetine capsules of Example 1 as a test product (“T”) and the commercial product CYMBALTA 60 mg capsules as a reference product (“R”), with healthy human volunteers in fasted and fed states, and plasma concentrations of the drug compounds were determined at intervals after dosing.

The following parameters were calculated:

AUC₀₋ₜ=the area under plasma concentration versus time curve, from the time of administration to the last measurable concentration.

AUC_{0-∞}=area under the plasma concentration versus time curve, from the time of administration to infinity.

Cₘₐₓ=maximum plasma concentration.

The average pharmacokinetic parameters for each product were calculated and are summarized in the following tables:

1. Fasted Study:

| | Example 1 | Reference | |
|---|---|---|---|
| Parameter | (T) | (R) | (T ÷ R × 100) |
| AUC_{0-∞} (ng · hour/mL) | 548.846 | 554.943 | 98.90 |
| AUC₀₋ₜ (ng · hour/mL) | 535.894 | 543.439 | 98.61 |
| Cₘₐₓ (ng/mL) | 30.235 | 31.547 | 95.84 |

2. Fed Study:

| | Example 1 | Reference | |
|---|---|---|---|
| Parameter | (T) | (R) | (T ÷ R × 100) |
| AUC_{0-∞} (ng · hour/mL) | 713.427 | 705.546 | 101.12 |
| AUC₀₋ₜ (ng · hour/mL) | 688.961 | 685.589 | 100.49 |
| Cₘₐₓ (ng/mL) | 34.460 | 38.372 | 89.80 |

### Example 2

### Duloxetine Hydrochloride-Containing Enteric Coated Pellets

| | |
|---|---|
| Ingredient | Weight Percent |

CORE

| | |
|---|---|
| Nonpareil seeds (#30-#35 mesh) | 6.48 |
| Hydroxypropyl methylcellulose | 0.38 |
| Water* | q.s. |

DRUG LAYERING

| | |
|---|---|
| Duloxetine hydrochloride | 12.94 |
| Hydroxypropyl methylcellulose 5 cps | 1.92 |
| Water* | q.s. |

SUBCOATING

| | |
|---|---|
| Copovidone | 32.71 |
| Glycine | 19.58 |
| Talc | 12.92 |
| Water* | q.s. |

ENTERIC COATING

| | |
|---|---|
| Eudragit ® L30 D55 | 5.69 |
| Aquacoat ® ECD** | 5.69 |
| Talc | 0.9 |
| Acetyltributyl citrate | 0.79 |
| Isopropyl alcohol* | q.s. |
| Dichloromethane* | q.s. |

*Evaporates during processing.
**Aquacoat ECD is a 30 percent by weight aqueous dispersion of ethylcellulose

Manufacturing Process:

Seal Coating:

1) HPMC is dissolved in hot water maintained at about 60° C. The solution is stirred and allowed to attain room temperature.

2) The solution is coated onto sugar spheres using the Wurster technique in a fluid-bed processor.

Drug Coating:

3) HPMC 5 cps is dissolved in hot water maintained at about 60° C. The solution is stirred and allowed to cool to room temperature.

4) Duloxetine hydrochloride is added to the polymer solution under continuous stirring.

5) Drug-loading dispersion is coated onto seal-coated spheres using the Wurster technique in a fluid-bed processor.

6) Drug-loaded pellets are dried and sifted.

Subcoating:

7) Copovidone, glycine and talc are dispersed in water and a uniform and smooth sub-coating dispersion is formed.

8) Sub-coating dispersion is sprayed onto drug-loaded pellets using the Wurster technique in a fluid-bed processor.

9) Sub-coated pellets are dried and sifted.

Enteric Coating:

10) Eudragit L 30D55 and Aquacoat® ECD are dispersed in methylene chloride.

11) To the polymer dispersion, acetyltributyl citrate is added under continuous stirring.

12) Talc is milled with isopropyl alcohol in a colloid mill and the dispersion is added to the polymer solution under stirring.

13) Enteric-coated dispersion is sprayed on sub-coated pellets using the Wurster technique in a fluid-bed processor.

14) Enteric-coated pellets are dried and sifted.

### Example 3

### Duloxetine Hydrochloride-Containing Enteric-Coated Pellets

| | |
|---|---|
| Ingredient | Weight Percent |

CORE

| | |
|---|---|
| Nonpareil seeds (#30-#35 mesh) | 20.18 |
| Hydroxypropyl methylcellulose | 1.2 |
| Water* | q.s. |

DRUG LAYERING

| | |
|---|---|
| Duloxetine hydrochloride | 40.3 |
| Hydroxypropyl methylcellulose 5 cps | 5.99 |
| Water* | q.s. |

SUBCOATING

| | |
|---|---|
| Copovidone | 48.5 |
| Hydroxypropyl methylcellulose 5 cps | 8.38 |
| Hydroxypropyl cellulose (KLUCEL ™ LF) | 3.35 |
| Talc | 1.68 |
| Magnesium state | 3.35 |
| Water* | q.s. |

ENTERIC COATING

| | |
|---|---|
| HPMCP HP 55 | 13.56 |
| Talc | 1.11 |
| Acetyltributyl citrate | 0.9 |
| Isopropyl alcohol* | q.s. |
| Dichloromethane* | q.s. |

*Evaporates during processing.

Manufacturing procedure: similar to that of Example 2.

### Example 4

### Duloxetine Hydrochloride-Containing Enteric-Coated Tablets

| | |
|---|---|
| Ingredient | Weight Percent |

CORE

| | |
|---|---|
| Duloxetine hydrochloride | 20.3 |
| Lactose monohydrate | 10.2 |
| Microcrystalline cellulose | 10.1 |
| Glycine | 2.1 |
| Sodium starch glycolate | 2.8 |
| Hydroxypropyl methylcellulose 5 cps | 5.99 |
| Water* | q.s. |
| Lactose monohydrate | 10.2 |
| Microcrystalline cellulose | 10.08 |
| Sodium starch glycolate | 2.8 |
| Magnesium stearate | 0.51 |

SUBCOATING

| | |
|---|---|
| Copovidone | 1.5 |
| Hydroxypropyl cellulose (KLUCEL ™ LF) | 3.35 |
| Talc | 1.68 |
| Glycine | 1 |
| Water* | q.s. |

ENTERIC COATING

| | |
|---|---|
| HPMCP HP 55 | 13.56 |
| Talc | 2.11 |
| Acetyltributyl citrate | 1.4 |
| Ferric oxide yellow | 0.32 |
| Isopropyl alcohol* | q.s. |
| Dichloromethane* | q.s. |

*Evaporates during processing.

Manufacturing Procedure:

A. Core Tablets:

1) Sift separately duloxetine hydrochloride, lactose monohydrate, microcrystalline cellulose, glycine and sodium starch glycolate through an ASTM #40 mesh sieve.

2) Mix all of the intragranular ingredients in a high-shear mixer to form a homogenous dry mixture.

3) Dissolve hydroxypropyl methylcellulose 5 cps in water.

4) Granulate the dry mixture of step 2 using step 3 solution in a high-shear mixer granulator, then dry the granules and size by passing through a sieve. Sift separately extragranular lactose monohydrate, microcrystalline cellulose, sodium starch glycolate and magnesium stearate through an ASTM #40 mesh sieve, and blend.

6) Blend granules of step 4 with the mixture of step 5 in a low-shear mixer.

7) Blend the mixture of step 6 with magnesium stearate.

8) Compress the mixture of step 7 into core tablets.

B: Subcoating:

9) Prepare a subcoating dispersion by dispersing copovidone, hydroxypropyl cellulose, talc and glycine in water under constant stirring.

10) Coat the core tablets of step 8 with the subcoating dispersion.

C. Enteric Coating:

11) Dissolve HPMC HP-55 in isopropyl alcohol under constant stirring.

12) Add methylene chloride to the solution of step 11.

13) Add talc, ferric oxide yellow, and acetyltributyl citrate to the solution of step 12 under constant stirring.

14) Coat the subcoated tablets of step 10 with the dispersion of step 13.

### Example 5

### Duloxetine Hydrochloride-Containing Enteric-Coated Pellets

| | |
|---|---|
| Ingredient | Weight Percent |

CORE

| | |
|---|---|
| Nonpareil seeds (#30-#35 mesh) | 6.44 |
| Hydroxypropyl methylcellulose | 0.38 |
| Water* | q.s. |

DRUG LAYERING

| | |
|---|---|
| Duloxetine hydrochloride | 12.87 |
| Hydroxypropyl methylcellulose 5 cps | 1.91 |
| Water* | q.s. |

SUBCOATING

| | |
|---|---|
| Copovidone | 32.52 |
| Glycine | 19.45 |
| Talc | 12.85 |
| Water* | q.s. |

ENTERIC COATING

| | |
|---|---|
| HPMCP HP 55 | 11.34 |
| Talc | 0.88 |
| Acetyl tributyl citrate (ATBC) | 0.78 |
| Isopropyl alcohol* | q.s. |
| Dichloromethane* | q.s. |

FINISHING LAYER

| | |
|---|---|
| Hydroxypropyl methylcellulose | 0.19 |
| Talc | 0.19 |
| Titanium dioxide | 0.19 |
| Water* | q.s. |

*Evaporates during processing.

Manufacturing Process All of the solid ingredients are sifted through a #40 mesh sieve prior to formulation.

### Seal Coating of Nonpareil Seeds:

1) Hydroxypropyl methylcellulose is dissolved in water.

2) The non-pareil seeds are warmed at 45° C.

3) The coating solution is then sprayed onto the seeds in a Wurster coater.

4) Pellets are dried at 50° C.±5° C. for 30 minutes.

Drug Layering:

5) Duloxetine hydrochloride is added to water with constant stirring to form a smooth dispersion.

6) The duloxetine dispersion is then passed through a colloid mill for 3-5 minutes.

7) Add hydroxypropyl methylcellulose to water under constant stirring to form dispersion and add this dispersion to the drug dispersion.

8) Warm the seal coated pellets to 45° C. and spray the drug dispersion onto them.

9) The pellets are dried at 50° C.±5° C. for 30 minutes.

Subcoating:

10) All the three ingredients are separately added to water under constant stirring and then mixed together.

11) The dispersion is sprayed onto pre-warmed drug layered pellets.

12) The fluidization airflow is reduced and the pellets dried at 50° C.±5° C. for 30 minutes.

Enteric Coating:

13) Isopropyl alcohol is placed into a stainless steel pressure vessel and HPMC HP-55 is added to it under constant stirring.

14) Methylene chloride is added to this solution.

15) Talc and ATBC are added to this solution under constant stirring.

16) The enteric coating dispersion is then sprayed onto pre-warmed subcoated pellets.

17) After completion of coating, the fluidization air is reduced and the pellets are dried at 50° C.±5° C. for 30 minutes.

Finishing Layer:

18) Hydroxypropyl methylcellulose, talc and titanium dioxide are added separately to water under constant stirring and then mixed together.

19) The finishing layer dispersion is sprayed onto pre-warmed enteric-coated pellets and the pellets are dried at 50° C.±5° C. for 30 minutes.

21) The enteric-coated pellets are mixed with 1 percent by weight talc and filled into size ‘0’ elongated hard gelatin capsules.

### Example 6

### Duloxetine Hydrochloride-Containing Enteric-Coated Pellets

| | |
|---|---|
| Ingredient | Weight Percent |

CORE

| | |
|---|---|
| Non pareil seeds (#30-#35 mesh) | 8.16 |
| Hydroxypropyl methylcellulose | 0.48 |
| Water* | q.s. |

DRUG LAYERING

| | |
|---|---|
| Duloxetine hydrochloride | 16.29 |
| Hydroxypropyl methylcellulose 5 cps | 2.42 |
| Water* | q.s. |

SUBCOATING

| | |
|---|---|
| Copovidone | 41.19 |
| Talc | 16.27 |
| Water* | q.s. |

ENTERIC COATING

| | |
|---|---|
| HPMCP HP 55 | 13.32 |
| Talc | 1.04 |
| Acetyl tributyl citrate | 0.9 |
| Isopropyl alcohol* | q.s. |
| Dichloromethane* | q.s. |

*Evaporates during processing

Manufacturing procedure: similar to that of Example 5.

We claim:

1. A delayed release duloxetine formulation comprising: (a) a pharmacologically inert solid core; (b) a drug layer comprising duloxetine or a salt thereof disposed over the core; (c) an optional subcoating layer disposed over the drug layer; and (d) an outer enteric coating layer.

2. The delayed release duloxetine formulation of claim 1, further comprising at least one chemically reactive substance present in a drug layer or a subcoating layer.

3. The delayed release duloxetine formulation of claim 2, wherein a chemically reactive substance comprises an α-amino acid.

4. The delayed release formulation of claim 2, wherein a chemically reactive substance is present in an amount ranging from about 0.1% to about 20% by weight of the formulation.

5. The delayed release formulation of claim 1, wherein a subcoating layer comprises a copolymer of N-vinyl-2-pyrrolidone and vinyl acetate.

6. The delayed release formulation of claim 1, wherein a subcoating layer comprises at least one water-insoluble or water-soluble plasticizer, or a combination thereof.

7. The delayed release formulation of claim 1, wherein an enteric coating layer comprises at least one water-insoluble or water-soluble plasticizer, or a combination thereof.

8. The delayed release formulation of claim 1, wherein the formulation is in the form of multi-particulates filled into a capsule, or is a tablet.

9. The delayed release formulation of claim 8, wherein multi-particulates are in the form of a powder, granules, pellets, or minitablets.

10. The delayed release duloxetine formulation of claim 1 comprising: (a) a solid core comprising duloxetine hydrochloride; (b) an optional subcoating layer; and (c) an enteric coating layer; further comprising at least one chemically reactive substance, a plasticizer, or a combination thereof.

11. A process for preparing a delayed release pharmaceutical formulation comprising: (i) providing a pharmacologically inert core; (ii) coating the inert core with a composition comprising duloxetine hydrochloride; (iii) optionally, coating the product of (ii) with a subcoating layer; and (iv) coating the product of (ii) or (iii) with an enteric layer; wherein the formulation comprises an amino acid in at least one of a composition comprising duloxetine hydrochloride and a subcoating layer.

12. The process of claim 11, wherein a composition comprising duloxetine hydrochloride comprises an amino acid.

13. The process of claim 11, wherein a subcoating layer comprises an amino acid.

14. The process of claim 11, wherein a subcoating layer comprises a plasticizer.

15. The process of claim 11, wherein an enteric layer comprises a plasticizer.

16. A process for preparing a delayed release pharmaceutical formulation comprising: (i) providing a core comprising duloxetine hydrochloride; (ii) optionally, coating the core with a subcoating layer; and (iii) coating the product of (i) or (ii) with an enteric layer; wherein the formulation comprises at least one α-amino acid in at least one of a core and a subcoating layer.

17. The process of claim 16, wherein a core comprises an amino acid.

18. The process of claim 16, wherein a subcoating layer comprises an amino acid.

19. The process of claim 16, wherein a subcoating layer comprises a plasticizer.

20. The process of claim 16, wherein an enteric layer comprises a plasticizer.
